(19) United States
(12) Patent Application Publication
(10) Pub. No.: US 2021/0137825 A1
(43) Pub. Date: May 13, 2021

(54) SKIN CARE COMPOSITIONS AND METHODS FOR MAKING THE SAME

(71) Applicant: KuL, Inc., Rancho Santa Fe, CA (US)

(72) Inventors: Timothy MULCAHY, Toronto (CA); Susan RAFFY, Costa Mesa, CA (US)

(73) Assignee: KuL, Inc., Rancho Santa Fe, CA (US)

(21) Appl. No.: 17/091,166

(22) Filed: Nov. 6, 2020

Related U.S. Application Data
(60) Provisional application No. 62/932,541, filed on Nov. 8, 2019.

(51) Int. Cl. A61K 8/9789; A61K 8/9722; A61K 8/34; A61K 8/99; A61K 8/92; A61K 8/73; A61K 8/891; A61K 8/67; A61Q 19/10; A61K 8/06; A61K 8/60; A61K 8/31

(52) U.S. Cl. CPC A61K 8/9789; A61K 2800/591; A61K 8/347; A61K 8/99; A61K 8/922; A61K 8/731; A61K 8/891; A61K 8/678; A61Q 19/10; A61K 8/062; A61K 8/927; A61K 8/60; A61K 8/31; A61K 8/671; A61K 2800/10; A61K 2800/522; A61K 8/9722

(57) ABSTRACT

Provided herein are compositions for skin care comprising cannabidiol, probiotics, and microalgae, optionally in a particular weight ratio. Optionally the compositions further comprise one or more of an antioxidant, a cleansing agent, an alcohol, an oil, and/or a flower or seed extract.

### CROSS-REFERENCE TO RELATED APPLICATIONS

This application claims the benefit under 35 U.S.C. § 119(e) of U.S. Provisional Application No. 62/932,541, filed Nov. 8, 2019, which is hereby incorporated by reference in its entirety.

### BACKGROUND

There exist a variety of skin care creams, lotions, ointments, and other products for cleansing, moisturizing, maintaining, and/or otherwise treating human skin. These products are typically commercially available.

Currently available skin care products face conventional problems commonly associated with such products, including, for example, that they cause the skin to look and/or feel greasy or dry, induce acne break-outs, are too abrasive, and contain too many (or the wrong proportion or combination of) ingredients for a particular skin types. Depending on the particular skin care product, those skilled in the art will appreciate that there may be additional or alternative conventional problems specific to any particular formulation.

Recently, the health benefits of cannabidiol (“CBD”), particularly with regard to medical conditions unrelated to skin, have been studied. Similarly, probiotics—various strains of bacteria closely associated with the naturally occurring bacteria in the human gastrointestinal system—have also been studied, particularly with regard to their potential impact on digestive health. Microalgae, which includes unicellular species typically found in either freshwater and saltwater systems, have also been studied for their potential impact on the nutritional value of food products.

These three ingredients, however, can also be used in skin care products. Specifically, it would be beneficial to have a skin care composition comprising CBD, probiotics, and microalgae. Further, currently available skin care products do not include an optimized combination of CBD, probiotics, and microalgae, much less one which further overcomes the conventional problems associated with available skin care products.

Accordingly, it would be desirable to have compositions and methods for skin care that combine CBD, probiotics, and microalgae, in an optimized manner, and which further overcome these deficiencies in the prior art.

### DETAILED DESCRIPTION OF THE INVENTION

Embodiments of the invention comprise skin care compositions comprising CBD. CBD can be derived from either medical marijuana or hemp, though it does not have the psychoactive properties associated with tetrahydrocannabinol (“THC”), another compound in the same family of bi-cyclic and tri-cyclic compounds cannabinoid compounds. CBD has the chemical structure set forth below:

[chemical structure]

Embodiments of the invention further comprise skin care compositions comprising probiotics. Examples of probiotics that may be used in the skin care products of the present invention comprise, for example, Lactobacillus Ferment. However, those skilled in the art will appreciate that this embodiment is exemplary, and that other probiotics or types of probiotics may be used in the compositions and methods of the present invention.

Embodiments of the invention further comprise skin care compositions comprising microalgae and/or microalgae extracts. Examples of microalgae extracts that may be used in the skin care products of the present invention comprise, for example, Chlorella Vulgaris or Scenedesmus Rubescens extracts. However, those skilled in the art will appreciate that this embodiment is exemplary, and that other microalgae or types of microalgae may be used in the compositions and methods of the present invention. Those skilled in the art will further appreciate that embodiments of the invention are not limited specifically to microalgae and/or microalgae extracts, and may additionally or alternatively comprise skin care compositions comprising algae and/or algae extracts.

Embodiments of the invention comprise skin care compositions comprising a combination of CBD, Probiotics, and microalgae, and methods of treating skin using said compositions.

Those skilled in the art will appreciate that the skin care compositions of the present invention may comprise additional components, including ingredients with known antioxidant properties. Antioxidants can, for example, reduce skin inflammation, allow for skin repair, and correct visible damage to the skin. Examples of ingredients with antioxidant properties that may be used in the skin care products of the present invention comprise, for example, Silybum Marianum (Milk Thistle) Ethyl Ester, Sambucus Nigra Fruit (Elderberry) Extract, and Camellia Sinensis Leaf Extract (Green Tea). However, those skilled in the art will appreciate that this embodiment is exemplary, and that other antioxidants or extracts and/or compounds with antioxidant properties may be used in the compositions and methods of the present invention. Embodiments of the invention may comprise Pyrus Malus (Apple) Fruit Extract.

Those skilled in the art will appreciate that the skin care compositions of the present invention may comprise additional components, including oils. Oils can, for example, assist in the repair of dry or cracked skin. Examples of oils that may be used in the skin care products of the present invention comprise, for example Cannabis Sativa (Hemp) Seed Oil and Butyrospermum Parkii (Shea) Oil. However, those skilled in the art will appreciate that this embodiment is exemplary, and that other oils and/or extracts and/or compounds with related properties may be used in the compositions and methods of the present invention. Embodiments of the invention may comprise Oenothera Biennis (Evening Primrose) Oil, Calophyllum Inophyllum (Tamanu) Oil, Camellia Japonica (Tsubaki) Seed Oil, Pelargonium Graveolens (Geranium) Oil, Squalene, Prunus Amygdalus (Sweet Almond) Oil, Borago Officinalis (Borage) Oil, Officinalis (Rosemary) Leaf Oil, Rubus Idaeus (Raspberry) Seed Oil, Rosa Canina (Dog Rose) Fruit Oil, and/or Rosa Canina (Rose Hip) Fruit Oil.

Those skilled in the art will appreciate that the skin care compositions of the present invention may comprise additional components, including sugars and sugar derivatives (such as glycoproteins). Sugar or sugar derivatives are capable of, for example, attracting water, allowing the skin to stay moisturized and smooth. Examples of sugars that may be used in the skin care products of the present invention comprise, for example Trehalose and Galactoarabinan. However, those skilled in the art will appreciate that this embodiment is exemplary, and that other sugars, sugar derivatives, and/or extracts and/or compounds with related properties may be used in the compositions and methods of the present invention.

Those skilled in the art will appreciate that the skin care composition of the present invention may comprise additional components, including peptides such as Pseudoalteromonas Ferment Extract.

Those skilled in the art will appreciate that the skin care compositions of the present invention may comprise additional components, including vitamins and vitamin derivatives. Vitamins can, for example, increase the amount of collagen in the skin. Examples of vitamins or vitamin derivatives that may be used in the skin care products of the present invention comprise, for example retinol (derived from vitamin A). However, those skilled in the art will appreciate that this embodiment is exemplary, and that other vitamins, vitamin derivatives, and/or extracts and/or compounds with related properties may be used in the compositions and methods of the present invention. Embodiments of the invention may comprise panthenol (pro-vitamin B5) and/or Tocopheryl Acetate 9 (derived from vitamin E).

Those skilled in the art will appreciate that the skin care compositions of the present invention may comprise additional components, including cleansing agents. Embodiments of the invention further comprise sulfate-free cleansing agents. Examples of cleansing agents that may be used in the skin care products of the present invention comprise, for example Sodium C14-16 Olefin Sulfonate, Cocamidopropyl Hydroxysultaine, Sodium Cocoyl, Glycinate, Cocamide MIPA, Capryloyl/Caproyl Methyl Glucamide, and Lauroyl/Myristoyl Methyl Glucamide. However, those skilled in the art will appreciate that this embodiment is exemplary, and that other cleansing agents and/or compounds with related properties may be used in the compositions and methods of the present invention.

Embodiments of the present invention may comprise water.

Embodiments of the present invention may comprise alcohols, including, for example, diols (e.g., propanediol), triols, and other polyols (e.g., glycerin (glycerol)), as well as fatty alcohols (e.g., cetearyl alcohol, cetyl alcohol) and sugar alcohols (e.g., xylitol, sorbitol), aromatic alcohols (e.g., phenoxyethanol) and/or any other alcohol or alcohol derivative suitable for use in the present invention. Relatedly, embodiments of the present invention may comprise glycerin (a polyol) and/or glycerin derivatives (e.g., polyglyceryl-6 distearate, polyglyceryl-3 beeswax, ethylhexylglycerin), including esters derived from glycerin (e.g., Caprylic/Capric Triglycerides), and/or any other glycerin or glycerin derivative suitable for use in the present invention.

Embodiments of the present invention may comprise silicon and/or organosilicon compounds and/or derivatives (e.g., silica, dimethicone), and/or any other silicon or silicon derivative suitable for use in the present invention.

Embodiments of the present invention may comprise flower or seed extracts (e.g., Salvia Hispanica (Chia) Seed Extract, Althaea Officinalis (Marshmallow) root extract) and/or any other natural extract and/or derivative thereof suitable for use in the present invention. Embodiments of the invention may comprise Rosa Damascena (Rose) Extract, Spilanthes Acmella Flower Extract, and/or Prunus Armeniaca (Apricot) Seed Powder.

Embodiments of the present invention may comprise esters, hydrogenated oils, and/or derivatives thereof (e.g., Jojoba ester(s), Hydrogenated Ethylhexyl Olivate) and/or any other esters, hydrogenated oils, and/or derivatives thereof suitable for use in the present invention.

Embodiments of the present invention may comprise additional sugars, polysaccharides, and/or derivatives thereof, other than those previously described herein (e.g., xanthan gum, sclerotium gum, pullulan) and/or any other sugars, polysaccharides, and/or derivatives thereof suitable for use in the present

Embodiments of the present invention may comprise fats and/or derivatives thereof (e.g., lecithin) and/or any other fats and/or derivatives thereof suitable for use in the present invention.

Embodiments of the present invention may comprise salts and/or derivatives thereof (e.g., sodium phosphate) and/or any other salts and/or derivatives thereof suitable for use in the present invention.

Embodiments of the present invention may comprise emulsifying agents (e.g., acrylates crosspolymer-4, polysorbate 20) and/or any other emulsifying agents suitable for use in the present invention.

Embodiments of the present invention may comprise colorants (e.g., Chlorophyllin-Copper Complex) and/or any colorants suitable for use in the present invention.

Embodiments of the present invention may comprise ingredients for adding fragrance(s) (e.g., fragrance, natural fragrance) and/or any ingredients for adding fragrance(s) suitable for use in the present invention.

Embodiments of the present invention may comprise Hydrogenated Olive Oil Unsaponifiables.

Embodiments of the invention may comprise any of the ingredients listed in one or more of FIGS. 1A to 1U.

Those skilled in the art will appreciate that the skin care compositions of the present invention may comprise additional ingredients, including, for example, stabilizing agents, preservatives, binders, additives, non-active ingredients, emulsifying agents, foam boosting agents, surfactants, viscosity controlling agents, excipients, bulking agents, fillers, diluents, and the like.

The invention further encompasses, among other things, the following numbered embodiments:

Embodiment 1a. In an exemplary embodiment of the invention, a skin care composition comprises CBD, Silybum Marianum (Milk Thistle) Ethyl Ester, Cannabis Sativa (Hemp) Seed Oil, Butyrospermum Parkii (Shea) Oil, Trehalose, Galactoarabinan, Chlorella Vulgaris Extract, Scenedesmus Rubescens Extract, Sambucus Nigra Fruit (Elderberry) Extract, and Lactobacillus Ferment.

Embodiment 1b. In an exemplary embodiment of the invention, Embodiment 1a may further comprise water, Propanediol, Glycerin, Dimethicone, Cetearyl Alcohol, Polyglyceryl-6 Distearate, Cannabidiol (Hemp Extracts), Salvia Hispanica (Chia) Seed Extract, Camellia Sinensis Leaf (Green Tea) Extract, Jojoba Ester, Althaea Officinalis (Marshmallow) Root Extract, Polyglyceryl-3 Beeswax, Cetyl Alcohol, Xanthan Gum, Lecithin, Sclerotium Gum, Pullulan, Silica, Xylitol, Sodium Phosphate, Sorbitol, Natural Fragrance, Phenoxyethanol, and Ethylhexylglycerin.

Embodiment 2a. In an exemplary embodiment of the invention, a skin care composition comprises CBD, retinol, Cannabis Sativa (Hemp) Seed Oil, Butyrospermum Parkii (Shea) Oil, Camellia Sinensis Leaf Extract (Green Tea), Trehalose, Galactoarabinan, Scenedesmus Rubescens Extract, Lactobacillus Ferment, Pseudoalteromonas Ferment Extract, and Chlorella Vulgaris Extract.

Embodiment 2b. In an exemplary embodiment of the invention, Embodiment 2a may further comprise water, Polyglyceryl-6 Distearate, Cetearyl Alcohol, Propanediol, Dimethicone, Hydrogenated Ethylhexyl Olivate, Jojoba Esters, Hydrogenated Olive Oil Unsaponifiables, Xanthan Gum, Polyglyceryl-3 Beeswax, Cetyl Alcohol, Glycerin, Xylitol, Sodium Phosphate, Sorbitol, Chlorophyllin-Copper Complex, Phenoxyethanol, Ethylhexylglycerin, and Natural Fragrance.

Embodiment 3a. In an exemplary embodiment of the invention, a skin care composition comprises the foregoing components in the associated percentages by weight: CBD (about 0.4%), Silybum Marianum (Milk Thistle) Ethyl Ester (about 1%), Cannabis Sativa (Hemp) Seed Oil (about 2.5%), Butyrospermum Parkii (Shea) Oil (about 2.5%), Chlorella Vulgaris Extract (about 1%), Scenedesmus Rubescens Extract (about 1%), Lactobacillus Ferment (about 1%), water (about 67%), Propanediol (about 4%), Glycerin (about 4%), Dimethicone (about 3%), Cetearyl Alcohol (about 2%), Polyglyceryl-6 Distearate and Jojoba Ester and Polyglyceryl-3 Beeswax and Cetyl Alcohol (about 4%), Salvia Hispanica (Chia) Seed Extract and Trehalose and Galactoarabinan and Xylitol and Sodium Phosphate and Sorbitol (about 1%), Camellia Sinensis Leaf (Green Tea) Extract (about 0.1%), Althaea Officinalis (Marshmallow) Root Extract (about 1%), Silica (about 1%), Xanthan Gum and Lecithin and Sclerotium Gum and Pullulan (about 2%), Natural Fragrance (about 1%), and Phenoxyethanol and Ethylhexylglycerin (about 1%).

Embodiment 3b. In an exemplary embodiment of the invention, the skin care composition of Embodiment 3a further comprises the foregoing components in the associated percentages by weight: Sambucus Nigra Fruit (Elderberry) Extract (about <1%), Cannabidiol (Hemp Extracts) (about <1%).

Embodiment 4a. In an exemplary embodiment of the invention, a skin care composition comprises the foregoing components in the associated percentages by weight: CBD (about 0.4%), retinol (about 0.1%), Cannabis Sativa (Hemp) Seed Oil (about 2.5%), Butyrospermum Parkii (Shea) Oil (about 2%), Camellia Sinensis Leaf Extract (Green Tea) (about 0.1%), Scenedesmus Rubescens Extract (about 1%), Lactobacillus Ferment (about 1%), Chlorella Vulgaris Extract (about 1%), water (about 72%), Cetearyl Alcohol (about 4%), Propanediol (about 4%), Dimethicone (about 2%), Polyglyceryl-6 Distearate and Jojoba Esters and Polyglyceryl-3 Beeswax and Cetyl Alcohol (about 6%), Xanthan Gum (about 0.2%), Glycerin (about 2%), Chlorophyllin-Copper Complex (about 0.001%), Phenoxyethanol and Ethylhexylglycerin and Ethyl Lauroyl Arginate HCl (about 1%), and Natural Fragrance (about 0.5%).

Embodiment 4b. In an exemplary embodiment of the invention, the skin care composition of Embodiment 4a further comprises the foregoing components in the associated percentages by weight: Trehalose (about <1%), Galactoarabinan (about <1%), Hydrogenated Olive Oil Unsaponifiables (about <1%), Xylitol (about <1%), Sodium Phosphate (about <1%), and Sorbitol (about <1%).

Embodiment 4c. In an exemplary embodiment of the invention, the skin care composition of Embodiment 4b further comprises the foregoing components in the associated percentages by weight: Pseudoalteromonas Ferment Extract (about <1%), Hydrogenated Ethylhexyl Olivate (about <1%).

Embodiment 5a. In an exemplary embodiment of the invention, a skin care composition comprises CBD, Silybum Marianum (Milk Thistle) Ethyl Ester, Cannabis Sativa (Hemp) Seed Oil, Rosa Damascena (Rose) Extract, Lactobacillus Ferment, Oenothera Biennis (Evening Primrose) Oil, Calophyllum Inophyllum (Tamanu) Oil, Camellia Japonica (Tsubaki) Seed Oil, Rosa Canina (Rose Hip) Fruit Oil, Spilanthes Acmella Flower Extract, Algae Extract, and Pelargonium Graveolens (Geranium) Oil.

Embodiment 5b. In an exemplary embodiment of the invention, Embodiment 5a may further comprise Squalane, Prunus Amygdalus (Sweet Almond) Oil, Caprylic/Capric Triglycerides, Tocopheryl Acetate 9, Borago Officinalis (Borage), Oil, Rosmarinus Officinalis (Rosemary) Leaf Oil, Sambucus Nigra Fruit Extract, Rubus Idaeus (Raspberry) Seed Oil, Rosa Canina (Dog Rose) Fruit Oil.

Embodiment 5c. In an exemplary embodiment of the invention, a skin care composition comprises the listed components as shown and described in FIG. 12.

Embodiment 6a. In an exemplary embodiment of the invention, a skin care composition comprises the listed components in about the associated percentages by weight as shown and described in FIG. 10.

Embodiments of the invention further comprise an exemplary method of preparing the compositions described herein, as set forth, for example, in FIGS. 3 and 7.

Those skilled in the art will appreciate that embodiments of the present invention may comprise any of the forgoing ingredients combined with any other or others, in any such combination or proportion or amount such that allows for a skin care composition as described herein. Those skilled in the art will further appreciate that the skin care compositions of the present invention may be in any form suitable to administer the composition topically, such as, for example, a cream, lotion, serum, cleaner, moisturizer, and/or any other suitable medium.

Embodiments of the invention further comprise use of the compositions of the present invention to treat, clean, cleanse, smooth, brighten, protect, defend, maintain, strengthen, hydrate, moisturize, freshen, improve the complexion of, improve the coloring of, minimize wrinkles and/or lines on, otherwise improve the appearance of, and/or repair the skin.

Embodiments of the invention further comprise use of the compositions of the present invention in a treatment regimen with one or more other compositions of the present invention. Those skilled in the art will appreciate that the present invention provides for the use of two or more complementary compositions for the purpose of skin care. For instance, and by way of example only, the present invention comprises a treatment regimen using two skin care compositions: a first skin care composition being a moisturizer, which hydrates the skin, and a second skin care composition being a serum, which is subsequently applies to fortify and strengthen the moisturized skin. Additionally or alternatively, a first skin care composition may be intended for application during the day, and a second skin care composition may be intended for application at night.

With reference to FIG. 1B, in some embodiments the composition comprises one or more, for example, one, two, three, four, five, six, seven, or eight of Aqua/Water, Sodium C14-16 Olefin Sulfonate, Cocamidopropyl Hydroxysulfaine, Cocamide MIPA, Acrylates Crosspolymer-4, Capryloyl/Caprayl Methyl Glucamide, Lauroyl/Myristoyl Methyl Glucamide, Pyrus Malus (Apple) Fruit-Extract, Cannabidiol (Hemp Extract), Prunus Armeniaca (Apricot) Seed Powder, Lactobacillus Ferment, Camellia Sinensis (Green Tea) Leaf Extract, Panthenol (Pro-Vitamin B5) Glycerin, Polysorbate 20, Chlorophyllin-Copper Complex, Ethylhexylglycerin, Phenoxyethanol, Fragrance.

With reference to FIG. 1C, in some embodiments the composition comprises one or more, for example, one, two, three, four, five, six, seven, or eight of Aqua/Water, Sodium C14-16 Olefin Sulfonate, Cocamidopropyl Hydroxysultaine, Cocamide MIPA, Acrylates Crosspolymer-4, Capryloyl/Caproyl Methyl Glucamide, Lauroyl/Myristoyl Methyl Glucamide, Pyrus Malus (Apple) Fruit Extract, Cannabidiol (Hemp Extract), Prunus Armeniaca (Apricot) Seed Powder, Lactobacillus Ferment, Camellia Sinensis (Green Tea) Leaf Extract, Panthenol (Pro-Vitamin B5) Glycerin, Polysorbate 20, Chlorophyllin-Copper Complex, Ethylhexylglycerin, Phenoxyethanol, Fragrance.

With reference to FIG. 1D, in some embodiments the composition comprises one or more, for example, one, two, three, four, five, six, seven, or eight of Aqua/Water, Sodium C14-16 Olefin Sulfonate, Cocamidopropyl Hydroxysultaine, Cocamide MIPA, Acrylates Crosspolymer-4, Capryloyl/Caproyl Methyl Glucamide, Lauroyl/Myristoyl Methyl Glucamide, Pyrus Malus (Apple) Fruit Extract, Cannabidiol (Hemp Extract), Prunus Armeniaca (Apricot) Seed Powder, Lactobacillus Ferment, Camellia Sinensis (Green Tea) Leaf Extract, Panthenol (Pro-Vitamin B5) Glycerin, Polysorbate 20, Chlorophyllin-Copper Complex, Ethylhexylglycerin, Phenoxyethanol, Fragrance.

With reference to FIG. 1E, in some embodiments the composition comprises one or more, for example, one, two, three, four, five, six, seven, or eight of Water/Aqua, Propanediol, Cannabis Sativa (Hemp) Seed Oil*, Glycerin, Dimethicone, Butyrospermum Parkii (Shea) Oil, Cetearyl Alcohol, Polyglyceryl-6 Distearate, Cannabidiol (Hemp Extracts), Trehalose, Galactoarabinan, Silybum Marianum (Milk Thistle) Ethyl Ester, Chlorella Vulgaris Extract (Micro-Algae), Scenedesmus Rubescens Extract, Lactobacillus Ferment, Scenedesmus Rubescens Extract, Salvia Hispanica (Chia) Seed Extract, Camellia Sinensis Leaf (Green Tea) Extract, Jojoba Ester, Althaea Officinalis (Marshmallow) Root Extract, Polyglyceryl-3 Beeswax, Cetyl Alcohol, Xanthan Gum, Lecithin, Sclerotium Gum, Pullulan, Silica, Xylitol, Sodium Phosphate, Sorbitol, Natural Fragrance, Phenoxyethanol, Ethylhexylglycerin.

With reference to FIG. 1F, in some embodiments the composition comprises one or more, for example, one, two, three, four, five, six, seven, or eight of Water/Aqua Propanediol Cannabis Sativa (Hemp) Seed Oil*, Glycerin, Dimethlcone, Cetearyl Akohol Pakglyceryl Distearate, Cannabidiol (Hemp Extract), Trehalose Gaiactoarobinon, Chlorella Vulgaris Extract (microalgae), Scenedesmus Rubescens Extract, Lactobacillus Ferment, Scenedesmut Rubescens Extract, Salvia Hispanica (Chia) Seed Extract, Butyrospermum Parkii (Shea) Oil Camellia Sinensis Leaf (green Tea) Extract, Jojoba Ester, Althaea Officinalis (Marshmallow) Root Extract, Polyglyceryl-3 Beeswax, Cetyl Alcohol Xanthan Gum, Lecithin, Sclerotium Gum, Pullulan, Silica, Xylitol, Sodium Phosphate, Sorbitol Natural Fragrance, Phenoxyethanol Ethylhexylglycerin.

With reference to FIG. 1G, in some embodiments the composition comprises one or more, for example, one, two, three, four, five, six, seven, or eight of Water/Aqua, Propanediol, Cannabis Sativa (Hemp) Seed Oil, Glycerin, Dimethicone, Cetearyl Alcohol, Polyglyceryl-6 Distearate, Cannabidiol (Hemp Extract), Trehalose, Galactoarabinan, Chlorella Vulgaris Extract (Microalgae), Scenedesmus Rubescens Extract, Lactobacillus Ferment, Scenedesmus Rubescens Extract, Lactobacillus Ferment, Scenedesmus Rubescens Extract, Salvia Hispanica (Chia) Seed Extract, Camellia Sinensis Leaf (Green Tea) Extract, Jojoba Ester, Althaea Officinalis (Marshmallow) Root Extract, Polyglyceryl-3 Beeswax, Cetyl Alcohol, Xanthan Gum, Lecithin, Sclerotium Gum, Pullulan, Silica, Xylitol, Sodium Phosphate, Sorbitol, Natural Fragrance, Phenoxyethanol, Ethylhexylglycerin.

With reference to FIG. 1H, in some embodiments the composition comprises one or more, for example, one, two, three, four, five, six, seven, or eight of Squalane, Prunus Amygdalus (Sweet Almond) Oil, Rosa Damascena (Rose) Extract, Caprylic/Capric Triglycerides, Silybum Marianum (Milk Thistle) Ethyl Ester, Cannabis Sativa, Cannabidiol (Hemp Extract), Tocopheryl Acetate 9, Lactobacillus Ferment, Borago Officinalis (Borage) Oil, Oenothera Biennis (Evening Primrose) Oil, Rosmarinus Officinalis (Rosemary) Leaf Oil, Sambucus Nigra Fruit Extract, Calophyllum Inophyllum (Tamanu) Oil*, Camellia Japonica Seed Oil, Rubus Idaeus (Raspberry) Seed Oil, Rosa Canina (Dog Rose) Fruit Oil, Spilanthes Acmella Flower Extract, Algae Extract, Pelargonium Graveolens (Geranium) Oil.

With reference to FIG. 11, in some embodiments the composition comprises one or more, for example, one, two, three, four, five, six, seven, or eight of Amygdalus (Swept Almond) Oil, Capriylic/ed CBD), Rosa Damascena (Rose) Extract, Tocopheryl Acetate 9, Lactobacillus Ferment, Borago Officinalis (Borage) Oil, Oenothera Biennis (Evening Primrose) Oil, Rosmarinus Officinalis (Rosemary) Leaf Oil, Calophyllum Inophyllum (Tamanu) Oil*. Camellia Japonica Seed Oil, Rubus Idaeus (Raspberry) Seed Oil Rosa Canina Fruit Oil Spilanthes Acmella Flower Extract, Algae Extract, Pelargonium Graveolens (Geranium) Oil.

With reference to FIG. 1J, in some embodiments the composition comprises one or more, for example, one, two, three, four, five, six, seven, or eight of Squalane, Prunus Amygdalus (Sweet Almond) Oil, Caprylic/Capric Triglycerides, Silybum Marianum (Milk Thistle) Ethyl Ester, Cannabis Sativa*, Cannabidiol (Hemp derived CBD), Rosa Damascena (Rose) Extract, Tocopheryl Acetate 9, Lactobacillus Ferment, Borago Officinalis (Borage) Oil, Oenothera Biennis (Evening Primrose) Oil, Rosmarinus Officinalis (Rosemary) Leaf Oil, Calophyllum Inophyllum (Tamanu) Oil*, Camellia Japonica Seed Oil, Rubus Idaeus (Raspberry) Seed Oil, Rosa Canina Fruit Oil Spilanthes Acmella Flower Extract, Algae Extract, Pelargonium Graveolens (geranium) Oil Amygdalus (Swept Almond) Oil, Capriylic/ed CBD), Rosa Damascena (Rose) Extract, Tocopheryl Acetate 9, Lactobacillus Ferment, Borago Officinalis (Borage) Oil, Oenothera Biennis (Evening Primrose) Oil, Rosmarinus Officinalis (Rosemary) Leaf Oil, Calophyllum Inophyllum (Tamanu) Oil*. Camellia Japonica Seed Oil, Rubus Idaeus (Raspberry) Seed Oil Rosa Canina Fruit Oil Spilanthes Acmella Flower Extract, Algae Extract, Pelargonium Graveolens (Geranium) Oil.

With reference to FIG. 1K, in some embodiments the composition comprises one or more, for example, one, two, three, four, five, six, seven, or eight of Water/Aqua, Polyglyceryl-6 Distearate, Cetearyl Alcohol, Cannabis Sativa (Hemp) Seed Oil, Glycerin, Propanediol, Dimethicone, Butyrospermum Parkii (Shea) Oil, Hydrogenated Ethylhexyl Olivate, Jojoba Esters, Retinol, Cannabidiol (Hemp Extract), Chlorella Vulgaris Extract, Lactobacillus Ferment, Trehalose, Galactoarabinan, Scenedesmus Rubescens Extract, Hydrogenated Olive Oil Unsaponifiables, Camellia Sinensis Leaf Extract, Xanthan Gum, Polyglyceryl-3 Beeswax, Cetyl Alcohol, Glycerin, Xylitol, Sodium Phosphate, Sorbitol, Chlorophyllin-Copper Complex, Phenoxyethanol, Ethylhexylglycerin, Natural Fragrance.

With reference to FIG. 1L, in some embodiments the composition comprises one or more, for example, one, two, three, four, five, six, seven, or eight of Water/Aqua, Polyglyceryl-6 Distearate, Cetearyl Alcohol, Cannabis Sativa (Hemp) Seed Oil, Glycerin, Propanediol, Dimethicone, Butyrospermum Parkii (Shea) Oil, Hydrogenated Ethylhexyl Olivate, Jojoba Esters, Retinol, Cannabidiol (Hemp Extract), Chlorella Vulgaris Extract, Lactobacillus Ferment, Trehalose, Galactoarabinan, Scenedesmus Rubescens Extract, Hydrogenated Olive Oil Unsaponifiables, Camellia Sinensis Leaf Extract, Xanthan Gum, Polyglyceryl-3 Beeswax, Cetyl Alcohol, Glycerin, Xylitol, Sodium Phosphate, Sorbitol, Chlorophyllin Copper Complex, Phenoxyethanol, Ethylhexylglycerin, Natural Fragrance.

With reference to FIG. 1M, in some embodiments the composition comprises one or more of, for example, one, two, three, four, five, six, seven, or eight of Water/Aqua, Polyglyceryl-6 Distearate, Cetearyl Alcohol, Cannabis Sativa (Hemp) Seed Oil, Glycerin, Propanediol, Dimethicone, Butyrospermum Parkii (Shea) Oil, Hydrogenated Ethylhexyl Olivate, Jojoba Esters, Retinol, Cannabidiol (Hemp Extract), Chlorella Vulgaris Extract, Lactobacillus Ferment, Trehalose, Galactoarabinan, Scenedesmus Rubescens Extract, Hydrogenated Olive Oil Unsaponifiables, Camellia Sinensis Leaf Extract, Xanthan Gum, Polyglyceryl-3 Beeswax, Cetyl Alcohol, Glycerin, Xylitol, Sodium Phosphate, Sorbitol, Chlorophyllin Copper Complex, Phenoxyethanol, Ethylhexylglycerin, Natural Fragrance.

With reference to FIG. 1N, in some embodiments the composition comprises one or more of, for example, one, two, three, four, five, six, seven, or eight of Water/Aqua, Propanediol, Cetearyl Alcohol, Polyglyceryl-6 Distearate, Cannabis Sativa (Hemp) Seed Oil, Cannabidiol (Hemp Extract), Glycerin, Butyrospermum Parkii (Shea) Butter, Dimethicone, Menthol, Jojoba Esters, Capsicum Frutescens (Red Chili Pepper) Oleoresin, Eugenia Caryophyllus (Clove) Flower Oil, Melaleuca Alternifolia (Tea Tree) Oil, Mentha Piperita (Peppermint) Oil, Arnica Montana Flower Extract, Tocopherol, Lactobacillus Ferment Extract, Xanthan Gum, Polyglyceryl-3 Beeswax, Phenoxyethanol, Ethylhexylglycerin, Cetyl Alcohol, Ethyl Lauroyl Arginate HCL, Natural Green Color.

With reference to FIG. 1O, in some embodiments the composition comprises one or more of, for example, one, two, three, four, five, six, seven, or eight of Water/Aqua, Propanediol, Cetearyl Alcohol, Polyglyceryl-6 Distearate, Cannabis Sativa (Hemp) Seed Oil*, Cannabidiol (CBD Hemp Extracts), Glycerin, Butyrospermum Parkii (Shea) Butter, Dimethicone, Menthol, Jojoba Esters, Capsicum Frutescens (Red Chili Pepper) Oleoresin, Eugenia Caryophyllus (Clove) Flower Oil, Melaleuca Alternifolia (Tea Tree) Oil, Mentha Piperita (Peppermint) Oil, Arnica Montana Flower Extract, Tocopherol, Lactobacillus Ferment Extract, Xanthan Gum, Polyglyceryl-3 Beeswax, Phenoxyethanol, Ethylhexylglycerin, Cetyl Alcohol, Ethyl Lauroyl Arginate HCI, Natural Green Color.

With reference to FIG. 1P, in some embodiments the composition comprises one or more of, for example, one, two, three, four, five, six, seven, or eight of Water/Aqua, Propanediol, Cetearyl Alcohol, Polyglyceryl-6 Distearate, Cannabis Sativa (Hemp) Seed Oil*, Cannabidiol (CBD Hemp Extracts), Glycerin, Butyrospermum Parkii (Shea) Butter, Dimethicone, Menthol, Jojoba Esters, Capsicum Frutescens (Red Chili Pepper) Oleoresin, Eugenia Caryophyllus (Clove) Flower Oil, Melaleuca Alternifolia (Tea Tree) Oil, Mentha Piperita (Peppermint) Oil, Arnica Montana Flower Extract, Tocopherol, Lactobacillus Ferment Extract, Xanthan Gum, Polyglyceryl-3 Beeswax, Phenoxyethanol, Ethylhexylglycerin, Cetyl Alcohol, Ethyl Lauroyl Arginate HCL, Natural Green Color.

With reference to FIG. 1Q, in some embodiments the composition comprises one or more of Pectin, Glucose Syrup, Cannabidiol (Hemp Extract), Natural Flavors and Colorings, Purified Water, Sugar, Citric Acid.

With reference to FIG. 1R, in some embodiments the composition comprises one or more of Pectin, Glucose Syrup, Cannabidiol (Hemp CBD), Natural Flavors and Colorings, Purified Water, Sugar, Citric Acid.

With reference to FIG. 1S, in some embodiments the composition comprises one or more of Organic MCT Oil, Hemp Derived Full Spectrum Cannabidiol, Organic Sweet Orange (Citrus sinensis) essential oil, Orange Citron Terpene blend.

With reference to FIG. 1T, in some embodiments the composition comprises one or more of Organic MCT Oil, Cannabidiol (Hemp Extract), Organic Sweet Orange (Citrus sinensis) essential oil, Orange Citron Terpene blend.

With reference to FIG. 1U, in some embodiments the composition comprises one or more of Organic MCT Oil, Full Spectrum Cannabidiol (Hemp Extract), Organic Sweet Orange (Citrus sinensis) essential oil, Orange Citron Terpene blend.

While this invention has been described with an emphasis upon preferred embodiments, it will be obvious to those of ordinary skill in the art that variations in the preferred systems and methods may be used and that it is intended that the invention may be practiced otherwise than as specifically described herein. Accordingly, this invention includes all modifications encompassed within the spirit and scope of the invention as defined by the claims that follow.

FIG. 3 provides a method for preparing the composition of embodiment 1a and/or 1b or an embodiments provided herein comprising the steps of:
PHASE A: ADD DIW TO MAIN COMPOUNDING TANK (MCT} AND BEGIN MIXING. PHASE B: IN A SEPARATE MIXING VESSEL, PRE-MIX PHASE B MATERIALS WELL.
ADD PHASE B TO MCT VORTEX AND MIX WELL. MIX FOR 5 MINUTES. BEGIN TO HEAT TO 70-75 C.
PHASE C: IN A SEPARATE MIXING VESSEL, PRE-MIX PHASE C MATERIALS. BEGIN TO HEAT TO 70-75 C. ONCE PHASE C IS ADDED TO PHASE AB (MCT}
HOMOGENIZE 10 MINUTES AT TEMP. RETURN TO REGULAR MIXING. BEGIN COOLING TO 35-40 C. PHASE D: AT 35-40 C, ADD PHASED MATERIALS TO MCT VORTEX AND MIX
PHASE E: AT 35-40 C, ADD PHASE E MATERIAL TO MCT VORTEX AND MIX WELL.
PHASE F: AT 35-40 C, ADD PHASE F MATERIAL TO MCT VORTEX AND MIX WELL.
PHASE G: AT 35-40 C, ADD PHASE F MATERIALS TO MCT VORTEX AND MIX WELL.
   Wherein PHASES A, B, C, D, E, F, and G are as described in FIG. 2.

FIG. 11 provides a method for preparing the composition of embodiment 1a and/or 1b or an embodiments provided herein comprising the steps of:
PHASE A: ADD PHASE A MATERIALS TO MAIN COMPOUNDING TANK (MCT} AND BEGIN HEATING TO 80-85 C. BEGIN TO COOL TO 70-75 C DO NOT SHOCK COOL.
PHASE B: ADD PHASE MATERIAL TO MAIN COMPOUNDING TANK (MCT) AND MIX WELL.
PHASE C: SUBMIT SAMPLE TO QUALITY CONTROL FOR PRODUCTION SAMPLE APPROVAL.
   Wherein PHASES A, B, and C are as described in FIG. 10.

### BRIEF DESCRIPTION OF FIGURES

FIGS. 1A to 1U describe compositions in accordance with embodiments of the present invention, and further describe exemplary methods of treatment therewith.

FIG. 2 describes compositions in accordance with embodiments of the present invention.

FIG. 3 describes, with reference to FIG. 2, a method of preparing compositions in accordance with embodiments of the present invention.

FIG. 4 describes compositions in accordance with embodiments of the present invention.

FIG. 5 illustrates a certificate of analysis of a composition in accordance with embodiments of the present invention.

FIG. 6 describes compositions in accordance with embodiments of the present invention.

FIG. 7 describes, with reference to FIG. 6, a method of preparing compositions in accordance with embodiments of the present invention.

FIG. 8 describes compositions in accordance with embodiments of the present invention.

FIG. 9 illustrates a certificate of analysis of a composition in accordance with embodiments of the present invention.

FIG. 10 describes compositions in accordance with embodiments of the present invention.

FIG. 11 describes, with reference to FIG. 10, a method of preparing compositions in accordance with embodiments of the present invention.

FIG. 12 describes compositions in accordance with embodiments of the present invention.

FIG. 13 illustrates a certificate of analysis of a composition in accordance with embodiments of the present invention.

1. A composition comprising at least one cannabidiol or cannabidiol derivative, at least one probiotic, and at least one algae extract.

2. The composition of claim 1, wherein the composition is formulated for use in skin care.

3. The composition of claim 1, wherein the algae extract is a microalgae extract comprising one or more of Chlorella Vulgaris Extract or Scenedesmus Rubescens Extract.

4. The composition of claim 1, wherein the probiotics comprise Lactobacillus Ferment.

5. The composition of claim 1, further comprising Silybum Marianum (Milk Thistle) Ethyl Ester; Butyrospermum Parkii (Shea) Oil; trehalose; galactoarabinan, and Sambucus Nigra Fruit (Elderberry) Extract.

6. The composition of claim 5, further comprising water, propanediol, glycerin, dimethicone, cetearyl alcohol, polyglyceryl-6 distearate, hemp extracts, Salvia Hispanica (Chia) Seed Extract, Camellia Sinensis Leaf (Green Tea) Extract, Jojoba Ester, Althaea Officinalis (Marshmallow) Root Extract, Polyglyceryl-3 Beeswax, Cetyl Alcohol, Xanthan Gum, Lecithin, Sclerotium Gum, Pullulan, Silica, Xylitol, Sodium Phosphate, Sorbitol, Natural Fragrance, Phenoxyethanol, and Ethylhexylglycerin.

7. The composition of claim 1, further comprising: retinol; Butyrospermum Parkii (Shea) Oil; Camellia Sinensis Leaf Extract (Green Tea); trehalose; galactoarabinan, and Pseudoalteromonas Ferment Extract.

8. The composition of claim 7, further comprising water, Polyglyceryl-6 Distearate, Cetearyl Alcohol, Propanediol, Dimethicone, Hydrogenated Ethylhexyl Olivate, Jojoba Esters, Hydrogenated Olive Oil Unsaponifiables, Xanthan Gum, Polyglyceryl-3 Beeswax, Cetyl Alcohol, Glycerin, Xylitol, Sodium Phosphate, Sorbitol, Chlorophyllin-Copper Complex, Phenoxyethanol, Ethylhexylglycerin, and Natural Fragrance.

9. A method for preparing the composition of claim 1 as substantially set forth and described in FIG. 3.

10. A method for preparing the composition of claim 7 as substantially set forth and described in FIG. 3.

11. The composition of claim 1, wherein the weight ratio between the cannabidiol or cannabidiol derivative, probiotics, and microalgae is about 0.4:1:2.

12. The skin care composition of claim 1, further comprising one or more ingredients with antioxidant properties.

13. The skin care composition of claim 1, further comprising one or more cleansing agents.

14. The skin care composition of claim 1, further comprising one or more alcohols.

15. The skin care composition of claim 1, further comprising one or more oils.

16. The skin care composition of claim 1, further comprising one or more flower or seed extracts.

17. A skin care composition comprising, by weight:
about 67% water;
about 4% Propanediol;
about 4% Glycerin;
about 4% combined Polyglyceryl-6 Distearate, Jojoba Ester, Polyglyceryl-3 Beeswax and Cetyl Alcohol;
about 3% Dimethicone;
about 2.5% Cannabis Sativa (Hemp) Seed Oil;
about 2.5% Butyrospermum Parkii (Shea) Oil;
about 2% Cetearyl Alcohol;
about 2% combined Xanthan Gum, Lecithin, Sclerotium Gum and Pullulan;
about 1% Silybum Marianum (Milk Thistle) Ethyl Ester;
about 1% Chlorella Vulgaris Extract;
about 1% Scenedesmus Rubescens Extract;
about 1% Lactobacillus Ferment;
about 1% combined Salvia Hispanica (Chia) Seed Extract, Trehalose, Galactoarabinan, Xylitol, Sodium Phosphate, Sorbitol;
about 1% Althaea Officinalis (Marshmallow) Root Extract;
about 1% Silica;
about 1% Natural Fragrance;
about 1% combined Phenoxyethanol and Ethylhexylglycerin;
about 0.4% cannabidiol; and
about 0.1% Camellia Sinensis Leaf (Green Tea) Extract.

18. The composition of claim 17, further comprising:
about <1% Sambucus Nigra Fruit (Elderberry) Extract; and
about <1% Cannabidiol (Hemp Extracts).

19. A skin care composition comprising, by weight:
about 72% water;
about 6% combined Polyglyceryl-6 Distearate and Jojoba Esters and Polyglyceryl-3 Beeswax and Cetyl Alcohol;
about 4% Cetearyl Alcohol;
about 4% Propanediol;
about 2.5% Cannabis Sativa (Hemp) Seed Oil;
about 2% Butyrospermum Parkii (Shea) Oil;
about 2% Dimethicone;
about 2% Glycerin;
about 1% combined Phenoxyethanol and Ethylhexylglycerin and Ethyl Lauroyl Arginate HCl;
about 1% Scenedesmus Rubescens Extract;
about 1% Lactobacillus Ferment;
about 1% Chlorella Vulgaris Extract;
about 0.5% Natural Fragrance;
about 0.4% CBD;
about 0.2% Xanthan Gum;
about 0.1% Camellia Sinensis Leaf Extract (Green Tea);
about 0.1% retinol; and
about 0.001% Chlorophyllin-Copper Complex.

20. The composition of claim 19, further comprising:
about <1% Trehalose;
about <1% Galactoarabinan;
about <1% Hydrogenated Olive Oil Unsaponifiables;
about <1% Xylitol;
about <1% Sodium Phosphate; and
about <1% Sorbitol.

21. A skin care composition comprising CBD, Silybum Marianum (Milk Thistle) Ethyl Ester, Cannabis Sativa (Hemp) Seed Oil, Rosa Damascena (Rose) Extract, Lactobacillus Ferment, Oenothera Biennis (Evening Primrose) Oil, Calophyllum Inophyllum (Tamanu) Oil, Camellia Japonica (Tsubaki) Seed Oil, Rosa Canina (Rose Hip) Fruit Oil, Spilanthes Acmella Flower Extract, Algae Extract, and Pelargonium Graveolens (Geranium) Oil.

22. The composition of claim 21, further comprising Squalane, Prunus Amygdalus (Sweet Almond) Oil, Caprylic/Capric Triglycerides, Tocopheryl Acetate 9, Borago Officinalis (Borage), Oil, Rosmarinus Officinalis (Rosemary) Leaf Oil, Sambucus Nigra Fruit Extract, Rubus Idaeus (Raspberry) Seed Oil, Rosa Canina (Dog Rose) Fruit Oil.

23. A method for preparing the composition of claim 21 as substantially set forth and described in FIG. 11.

24. A skin care composition comprising, by weight:
about 67% squalane;
about 15% Prunus Amygdalus Dulcis (Sweet Almond) Oil;
about 2.5% Caprylic/Capric Triglycerides;
about 2.5% Silybum Marianum (Milk Thistle) Ethyl Ester;
about 0.05% Tocopherol;
about 0.05% Tocopherol Acetate;
about 2% Cannabis Sativa (Hemp) Seed Oil;
about 2% CBD;
about 0.05% Borago Officinalis (Borage) Oil;
about 0.05% Oenothera Biennis (Evening Primrose) Oil;
about 1% Sambucus Nigra Fruit Extract;
about 0.001% Rosemarinus Officinalis (Rosemary) Leaf Oil;
about 0.001% Calophyllum Inophyllum (Tamanu) Oil;
about 0.1% Camellia Oleifera Seed Oil;
about 0.1% Rubus Idaeus (Raspberry) Seed Oil;
about 0.1% Rosa Canina Fruit Oil;
about 2% combined Caprylic/Capric Triglyceride (and) Spilanthes Acmella Flower Extract;
about 0.1% Chlamydomonas Reinhardtii Extract;
about 0.001% Pelargonium Graveolens Leaf Oil;
about 0.001% Lactobacillus Ferment; and
about 5% Rosa Damascena (Rose) Extract.
